(19) United States
(12) Patent Application Publication
(10) Pub. No.: US 2017/0164961 A1
(43) Pub. Date: Jun. 15, 2017

(54) CORACOID DRILL GUIDE AND METHOD OF USE THEREOF

(71) Applicant: Smith & Nephew, Inc., Memphis, TN (US)

(72) Inventors: Daniel Haberman, Boston, MA (US); Stephen A. Santangelo, Sturbridge, MA (US); Mason J. Bettenga, Memphis, TN (US); George S. Athwal, London (CA)

(73) Assignee: Smith & Nephew, Inc., Memphis, TN (US)

(21) Appl. No.: 15/373,619

(22) Filed: Dec. 9, 2016

Related U.S. Application Data
(60) Provisional application No. 62/266,088, filed on Dec. 11, 2015.

(51) Int. Cl. A61B 17/17; A61B 17/16

(52) U.S. Cl. CPC A61B 17/1778; A61B 17/1684

(57) ABSTRACT

A coracoid drill guide including a hollow shaft for passage of a drill sleeve. The shaft is fitted with a cap having a distally-extending finger. A longitudinal axis of the finger is offset from the longitudinal axis of the shaft by a first fixed distance. The shaft is disposed within a first cannulation of sliding and rotating block that has two cannulations at a second fixed distance apart. The drill guide ensures that a first drill hole will always be placed at the first fixed distance from both the distal tip and the lateral edge of the coracoid, and that a second drill hole will always be placed at the first fixed distance from the lateral edge of the coracoid and at the second fixed distance from the first drill hole.

### CROSS-REFERENCE TO RELATED APPLICATIONS

This application claims priority to and benefit of co-pending U.S. Provisional Patent Application No. 62/266,088, filed Dec. 11, 2015, entitled CORACOID DRILL GUIDE AND METHOD OF USE, the contents of which are incorporated by reference herein in their entirety for all purposes.

### FIELD

The present disclosure relates generally to surgical drill guides. More specifically, the present disclosure relates to surgical drill guides for positioning and orienting of holes to be drilled in a coracoid bone.

### BACKGROUND

The shoulder joint, also referred to as the glenohumeral joint, is the joint between the glenoid cavity (a part of the scapula) and the head of the humerus (upper arm bone). The glenoid cavity is shallow, covering only about a third of the head humeral head. As a result, the glenoid cavity provides relatively little bony constraint upon motion of the humerus and the glenohumeral joint exhibits the widest range of motion of all joints in the human body. While the glenohumeral joint is also constrained by soft tissue (e.g., cartilage attached to the rim of the glenoid cavity, tendons, etc.), in general, soft tissue cannot provide the same degree of constraint as bone. Accordingly, it is relatively easy to force the humerus from its normal anatomical position with respect to the glenoid socket and dislocate the shoulder. While not life threatening, a dislocated shoulder can cause pain and immobilization of the joint, impacting a patient's lifestyle.

In the case of severe bone loss, a surgeon may perform a “Latarjet procedure” to repair glenohumeral instability. In a Latarjet procedure, a surgeon attempts to restore bone mass to the glenoid cavity by securing a bone graft to a portion of the patient's scapula referred to as the coracoid process, or simply coracoid. A drill guide assists the surgeon in judging where to drill passages in the coracoid and helps to keep the drill piece steady during the drilling process. In an open surgical procedure, the passages can be drilled free-hand. However, as arthroscopic surgeries become more common, drilling passages at a fixed distance apart and at a fixed distance from the lateral edge of the coracoid presents challenges since the anatomy and geometry of the coracoid varies among individuals and often has a curved shape. Misalignment of the drill holes can result in a coracoid fragment that can project from the glenoid surface, or drill holes that break through the lateral cortex of the coracoid.

### SUMMARY

Described herein is a coracoid drill guide that allows for the measured placement of drill holes or passages in a consistent and reliable fashion. The drill guide of this disclosure consists of a hollow shaft for passage of a drill sleeve. The shaft is fitted with a cap having a distally-extending finger. A longitudinal axis of the finger is offset from the longitudinal axis of the shaft by a first fixed distance. The shaft is disposed within a first cannulation of sliding and rotating block that has two cannulations at a second fixed distance apart. In allowing this free rotation of both the block and the shaft, the drill guide of this disclosure ensures that a first drill hole will always be placed at a the first fixed distance from both the distal tip and the lateral edge of the coracoid. The drill guide also ensures that a second drill hole will always be placed at the first fixed distance from the lateral edge of the coracoid and at the second fixed distance from the first drill hole. Advantageously, the coracoid drill guide of this disclosure allows offset measurements in two directions with freedom of rotation to ensure perpendicular placement of the drill holes.

In examples, the coracoid drill guide of this disclosure includes a generally rectangular body having a proximal end and a distal end. The body has first and second parallel cannulations extending from the proximal end to the distal end and separated by a first distance. The coracoid drill guide also includes an elongate, hollow shaft configured for passage of a drill sleeve. The shaft is slidably and rotably disposed within the first cannulation of the body. A distal end of the shaft has a projecting member, a longitudinal axis of the projecting member being offset from a longitudinal axis of the shaft by a second distance. In further examples, the first distance is about 10 mm and the second distance is about 5 mm.

Examples of a method of drilling a passage in a coracoid of this disclosure include: 1) placing a lateral surface of a projection member of a shaft into contact with a distal tip of a patient's coracoid process, the shaft being slidably and rotatably disposed within a first cannulation of a body of a drill guide; 2) rotating the shaft to place the lateral surface of the projection member into contact with a lateral surface of the coracoid; and 3) passing a drill having a first drill sleeve through the shaft to drill a first passage in the coracoid. A longitudinal axis of the projecting member is offset from a longitudinal axis of the shaft by a first distance and the first passage is positioned at the first distance from both the lateral edge and the distal tip of the coracoid. In further examples, the first distance is about 5 mm. In examples, the method further includes retracting the drill guide from the first drill sleeve.

In other examples, the method further includes: 1) inserting the first drill sleeve into a second cannulation of the body of the drill guide; 2) rotating the body so that the shaft is substantially parallel to the first drill sleeve on the opposite side of the drill sleeve from the distal tip of the coracoid; 3) placing the lateral surface of the projection member in contact with the lateral edge of the coracoid; and 4) passing a drill having a second drill sleeve through the shaft to drill a second passage in the coracoid. The second passage is positioned at the first distance from the lateral edge of the coracoid and at a second distance from the first passage. In examples, the second distance is about 10 mm. The first passage and the second passage are substantially parallel. In other examples, the method further includes retracting the drill guide from the second drill sleeve.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The disclosure will be more fully understood by reference to the detailed description, in conjunction with the following figures, wherein:

FIG. 1 is an illustration of an exemplary coracoid drill guide of this disclosure;

FIGS. 2-3 illustrate a method of using the coracoid drill guide of FIG. 1 for a single point of fixation; and

FIGS. 4-5 illustrate a method of using the coracoid drill guide of FIG. 1 for a second point of fixation.

### DETAILED DESCRIPTION

In the description that follows, like components have been given the same reference numerals, regardless of whether they are shown in different examples. To illustrate example(s) in a clear and concise manner, the drawings may not necessarily be to scale and certain features may be shown in somewhat schematic form. Features that are described and/or illustrated with respect to one example may be used in the same way or in a similar way in one or more other examples and/or in combination with or instead of the features of the other examples.

Comprise, include, and/or plural forms of each are open ended and include the listed parts and can include additional parts that are not listed. And/or is open ended and includes one or more of the listed parts and combinations of the listed parts.

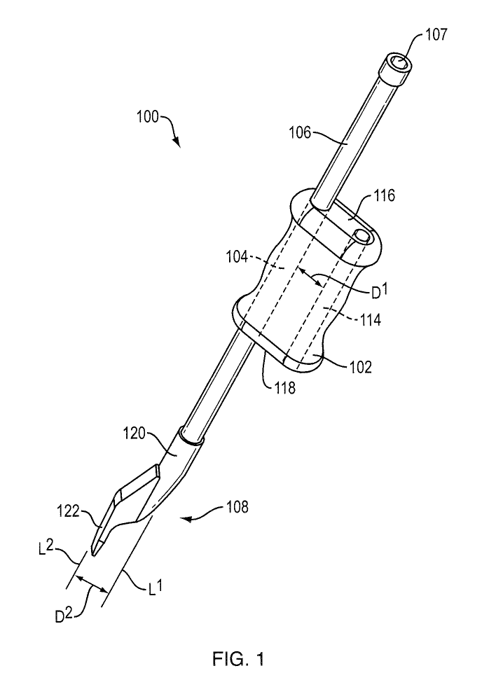

Turning now to FIG. 1, an exemplary coracoid drill guide 100 of this disclosure is shown. The drill guide 100 generally includes a rectangular guide block 102 and an elongate, hollow shaft 106. The guide block 102 has a first cannulation 104 and a second cannulation 114 extending through the guide block 102 from a proximal end 116 to a distal end 118. The first and second cannulations 104, 114 extend substantially parallel to each other and are separated by a first offset distance D¹. In examples, the first offset distance D¹ may be about 10 mm, although other suitable distances are contemplated by this disclosure. The guide block 102 is preferably made of stainless steel. However, other suitable materials, including other metals and plastics, are contemplated by this disclosure.

The shaft 106 comprises a bore 107 extending along a first longitudinal axis L¹ of the shaft 106 and configured to allow the passage of a drill sleeve during a drilling procedure. In examples, a diameter of the bore 107 is about 3 mm. The shaft 106 is both slidably and rotationally disposed within the first cannulation 104 of the guide block 102. A distal end of the shaft 106 includes projection member 108, the use of which will be described in more detail below. The projection member 108 comprises a cap 120 fixedly attached to the shaft 106 and a distally-extending finger 122 fixedly mounted to a distal surface of the cap 120. The finger 22 extends along a second longitudinal axis L². Both of the projection member 108 and the shaft 106 are preferably made of stainless steel, although other suitable materials, including other metals and plastics, are contemplated by this disclosure. In FIG. 1, it can be seen that the second longitudinal axis L² of the finger is offset from the first longitudinal axis L¹ of the shaft 106 by a second offset distance D². In examples, the second offset distance D² may be about 5 mm. However, other suitable offset distances D² are contemplated by this disclosure.

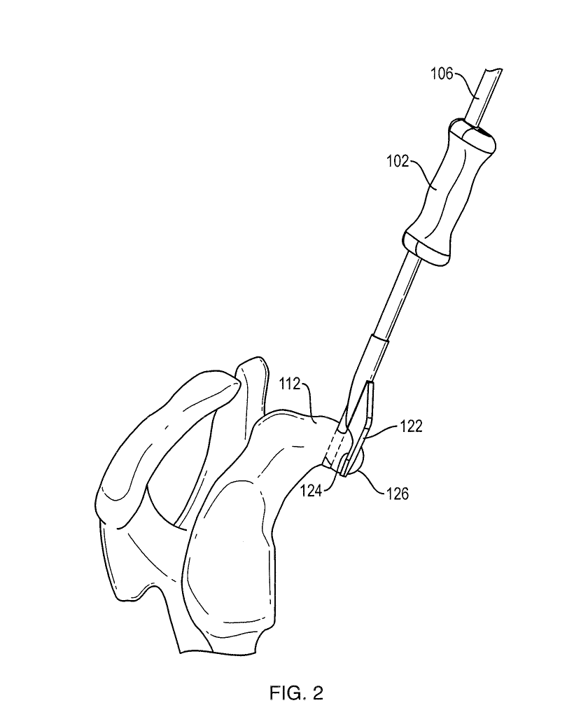

FIGS. 2-5 illustrate a method of using the drill guide 100 during a surgical procedure, for instance, a Latarjet procedure. However, the drill guide 100 could be scaled and used in any method or procedure that would require offset measurements in two directions with freedom of rotation to ensure perpendicular placement of drill holes or passages. For ease of illustration, the soft tissue (e.g., muscle, cartilage, tendons) is not shown in the drawings.

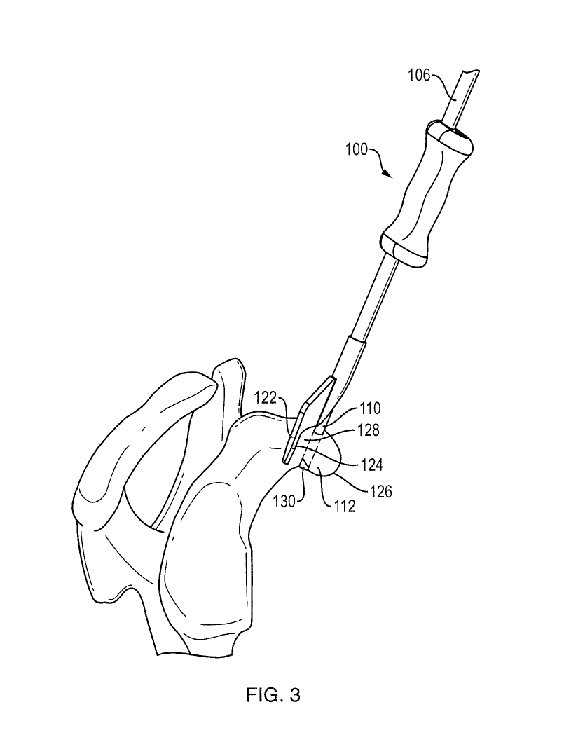

FIG. 2 illustrates the bony anatomy of a patient's shoulder, including the coracoid 112. The shaft 106 extending through the guide block 102 is positioned with a lateral surface 124 of the finger 122 into contact with the distal tip 126 of the coracoid 112. This offsets the cannulated shaft 106 from the distal tip 126 of the coracoid 112 by the second offset distance D² (i.e., about 5 mm). Next, as shown in FIG. 3, the shaft 106 is rotated clockwise to place the lateral surface 124 of the finger 122 in contact with the lateral edge 128 of the coracoid 112. In this manner, the cannulated shaft 106 is now positioned at the second offset distance D² from both the lateral edge 128 and the distal tip 126 of the coracoid 112 (i.e., about 5 mm). A drill (for example, a 2.8 mm drill) having a first drill sleeve 110 is then advanced through the shaft 106 to drill a first passage 130 through the coracoid 112. Thus, the first passage 130 is now positioned at the second offset distance D² from both the lateral edge 128 and the distal tip 126 of the coracoid 112 (i.e., about 5 mm). The drill guide 100 is then retracted from the drill sleeve 110, optionally leaving the drill sleeve 110 in place within the first passage 130 in the coracoid 112.

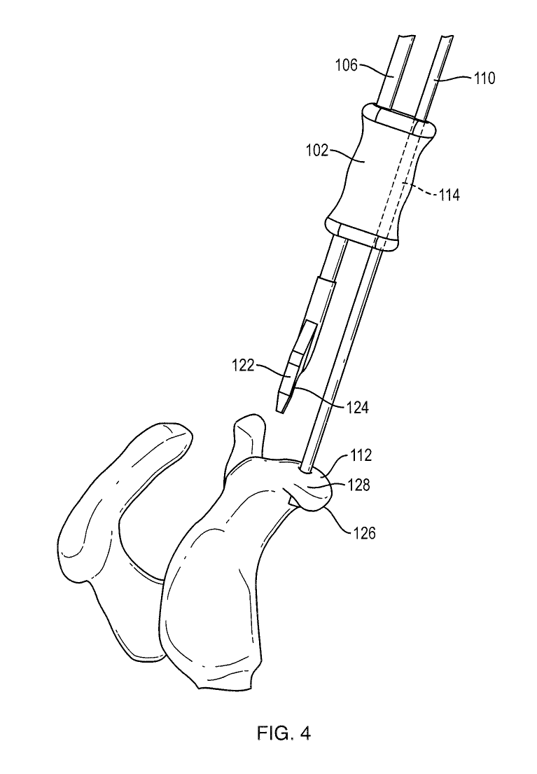

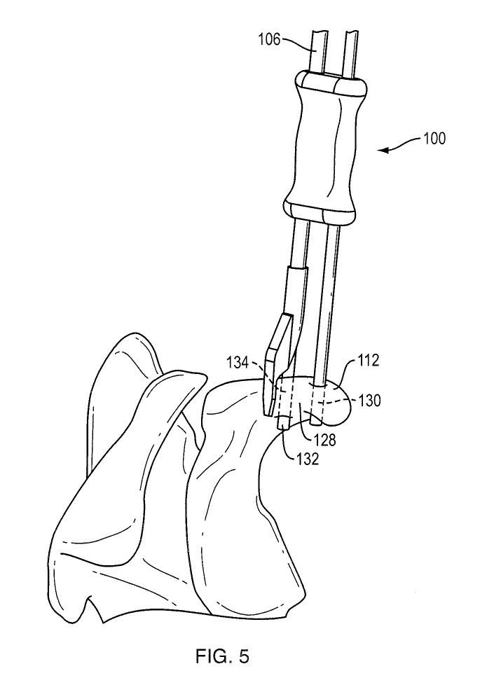

Turning now to FIG. 4, if a second point of fixation is desired, the drill sleeve 110 is subsequently inserted into the second cannulation 114 of the guide block 102. The guide block 102 is rotated clockwise so that the shaft 106 is substantially parallel to the drill sleeve 110 on the opposite side of the drill sleeve 110 from the distal tip 126. The lateral surface 124 of the finger 122 is again placed in contact with the lateral edge 128 of the coracoid 112. In this manner, the shaft 106 is positioned at the first offset distance D¹ from the drill sleeve 110 (i.e, about 10 mm) and at the second offset distance D² from the lateral edge 128 of the coracoid 112 (i.e., about 5 mm). In FIG. 5, a drill with a second drill sleeve 132 then is placed through the shaft 106 for drilling a second passage 134 in the coracoid 112. The drill guide 100 is then retracted from the second drill sleeve 132. Thus, the second passage 134 is now positioned at the first offset distance D¹ from the first passage 130 (i.e, about 10 mm) and at the second offset distance D² from the lateral edge 128 of the coracoid 112 (i.e., about 5 mm).

Based on the description above, it will be appreciated that use of the drill guide 100 of this disclosure advantageously ensures that the first passage 130 is always placed at a fixed distance from both the distal tip 126 and the lateral edge 128 of the coracoid 112. The drill guide 100 of this disclosure also advantageously ensures that the second passage 134 will always be placed at a fixed distance from both the first passage 130 and the lateral edge 128 of the coracoid 112. Moreover, the drill guide 100 ensures that the first passage 130 and the second passage 134 are always parallel.

One skilled in the art will realize the disclosure may be embodied in other specific forms without departing from the spirit or essential characteristics thereof. The foregoing examples are therefore to be considered in all respects illustrative rather than limiting of the disclosure described herein. Scope of the disclosure is thus indicated by the appended claims, rather than by the foregoing description, and all changes that come within the meaning and range of equivalency of the claims are therefore intended to be embraced therein.

What is claimed is:

1. A drill guide comprising:
a generally rectangular body having a proximal end and a distal end, the body including first and second parallel cannulations extending from the proximal end to the distal end and separated by a first distance;
an elongate, hollow shaft configured for passage of a drill sleeve, the shaft slidably and rotably disposed within the first cannulation of the body, a distal end of the shaft comprising a projecting member;
wherein a longitudinal axis of the projecting member is offset from a longitudinal axis of the shaft by a second distance.

2. The drill guide of claim 1, wherein the first distance is about 10 mm.

3. The drill guide of claim 1, wherein the second distance is about 5 mm.

4. A method of drilling a passage in a coracoid, the method comprising:
placing a lateral surface of a projection member of a shaft into contact with a distal tip of a patient's coracoid process, the shaft being slidably and rotatably disposed within a first cannulation of a body of a drill guide;
rotating the shaft to place the lateral surface of the projection member in contact with a lateral surface of the coracoid; and
passing a drill having a first drill sleeve through the shaft to drill a first passage in the coracoid;
wherein a longitudinal axis of the projecting member is offset from a longitudinal axis of the shaft by a first distance and the first passage is positioned at the first distance from both the lateral edge and the distal tip of the coracoid.

5. The method of claim 4, wherein the first distance is about 5 mm.

6. The method of claim 4, further comprising retracting the drill guide from the first drill sleeve.

7. The method of claim 4, further comprising:
inserting the first drill sleeve into a second cannulation of the body of the drill guide;
rotating the body so that the shaft is substantially parallel to the first drill sleeve on the opposite side of the drill sleeve from the distal tip of the coracoid;
placing the lateral surface of the projection member into contact with the lateral edge of the coracoid;
passing a drill having a second drill sleeve through the shaft to drill a second passage in the coracoid;
wherein the second passage is positioned at the first distance from the lateral edge of the coracoid and at a second distance from the first passage.

8. The method of claim 7, wherein the first distance is about 5 mm.

9. The method of claim 7, wherein the second distance is about 10 mm.

10. The method of claim 7, wherein the first passage and the second passage are substantially parallel.

11. The method of claim 7, further comprising retracting the drill guide from the second drill sleeve.
